(19) United States
(12) Patent Application Publication
(10) Pub. No.: US 2016/0157527 A1
(43) Pub. Date: Jun. 9, 2016

(54) BREAST SUPPORT

(71) Applicant: Jody Harbour, Oakville (CA)

(72) Inventor: Jody Harbour, Oakville (CA)

(21) Appl. No.: 14/958,480

(22) Filed: Dec. 3, 2015

Related U.S. Application Data
(60) Provisional application No. 62/086,972, filed on Dec. 3, 2014.

(51) Int. Cl. A41C 3/00; A61F 2/78; A41C 1/06

(52) U.S. Cl. CPC A41C 3/0021; A41C 1/06; A41C 3/0064; A61F 2/78; A61F 2002/7862

(57) ABSTRACT

A garment for use by a woman having a pair of breasts is disclosed. The garment comprises a central portion, a harness and a pair of support portions. The central portion is disposed between said pair of breasts in use. The harness includes a girdle portion which encircles a midriff of the woman in use in a manner which resists upward movement, a pair of straps from which the central portion is suspended in use and which extends from the central portion, over the shoulders of the woman, to the girdle portion. Each support portion is disposed beneath a respective one of the pair of breasts in use and extends from the central portion to the harness.

### CROSS-REFERENCE TO RELATED APPLICATIONS

This Application claims the benefit of U.S. Provisional Application 62/086,972 filed on Dec. 3, 2014, the contents of which are incorporated herein in its entirety.

### FIELD OF THE INVENTION

The present invention relates to the field of breast support garments.

### BACKGROUND OF THE INVENTION

Over the past century, countless innovations in brassiere design have been both patented and commercialized, many having the objectives of providing improved breast appearance. Despite the large number of brassiere designs available on the market there is a remarkable absence of support garments designed with comfort in mind.

### SUMMARY OF THE INVENTION

The present invention teaches a garment for use by a woman having a pair of breasts forms one aspect of the invention. This garment comprises a central portion, a harness and a pair of support portions. The central portion is disposed between said pair of breasts in use. The harness includes: a girdle portion which encircles the midriff of said woman in use in a manner which resists upward movement; and a pair of straps from which the central portion is suspended in use and which, in use, extend from the central portion, over the shoulders of said woman, to the girdle portion. The support portions are disposed beneath respective breasts in use and each extend from the central portion to the harness.

According to another aspect of the invention, the central portion can be a pair of spacers, each spacer being defined by an extension of a respective support portion.

According to another aspect of the invention, the spacers can be secured together in use.

According to another aspect of the invention, the support portions can be cylindrical and each spacer can be defined by a cylindrical extension of a respective support portion.

According to another aspect of the invention, the central portion can be cushioned and space the breasts apart from one another.

According to another aspect of the invention, the support portions can be cushioned and support the undersides of the breasts.

According to another aspect of the invention, the cushions of the spacers and of the support portions can be removable, for laundering of the garment.

Garments according to the invention can, according to another aspect of the invention, form part of garment systems.

One system includes a garment and a gel pack adapted to take the place of one of the cushions, for providing temperature therapy to the wearer.

Another system comprises the garment and a prosthetic which defines one of the breasts of said woman in use and which is suspended from the harness.

Other advantages of the invention will become apparent to persons of ordinary skill in the art upon review of the appended claims, upon review of the following detailed description and upon review of the enclosed drawings, the latter being briefly described hereinafter.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is an exploded view of the components of a garment according to an exemplary embodiment of the present invention;

FIG. 2 is a view of foam inserts for use with the structure of FIG. 1;

FIG. 3 is a view of a gel insert for use with the structure of FIG. 1;

FIG. 4 is a schematic perspective view of the components of FIG. 1 assembled for use;

FIG. 5 is a view of the components of FIG. 1, and bracketed components B of FIG. 2, positioned on a mannequin in an environmental depiction;

FIG. 6 is a rear view of the garment structure of FIG. 5;

FIG. 7A is a front view of the components of FIG. 1, and bracketed components B of FIG. 2, in use;

FIG. 7B is a view similar to FIG. 7A, from a rotated vantage point;

FIG. 8 is a front view of the components of FIG. 1, and bracketed components A of FIG. 2, in use;

FIG. 9 is a back view of the structure of FIG. 8;

FIG. 10 is a view similar to FIG. 8;

FIG. 11 is a view of a prosthetic system according to a related variant of the present invention;

FIG. 12 is a view of portions of the system of FIG. 1 and encircled structure 12 of FIG. 11, positioned upon a mannequin and in a single breast underside supporting application;

FIG. 13 is a view of the structure of FIG. 12 in use with encircled structure 13 of FIG. 11 in a further application;

FIG. 14 is a view of portions of the system of FIG. 1 and the structure of FIG. 11 in use on a mannequin according to a yet further related environmental application;

FIG. 15 is a front view of a system according to another embodiment of the invention;

FIG. 16 is a rear view of the system of FIG. 15;

FIG. 17 is a profile perspective view of the system of FIG. 15; and

FIG. 18 is a view, similar to FIG. 15, in a partially disassembled configuration.

### DETAILED DESCRIPTION OF THE PREFERRED EMBODIMENTS

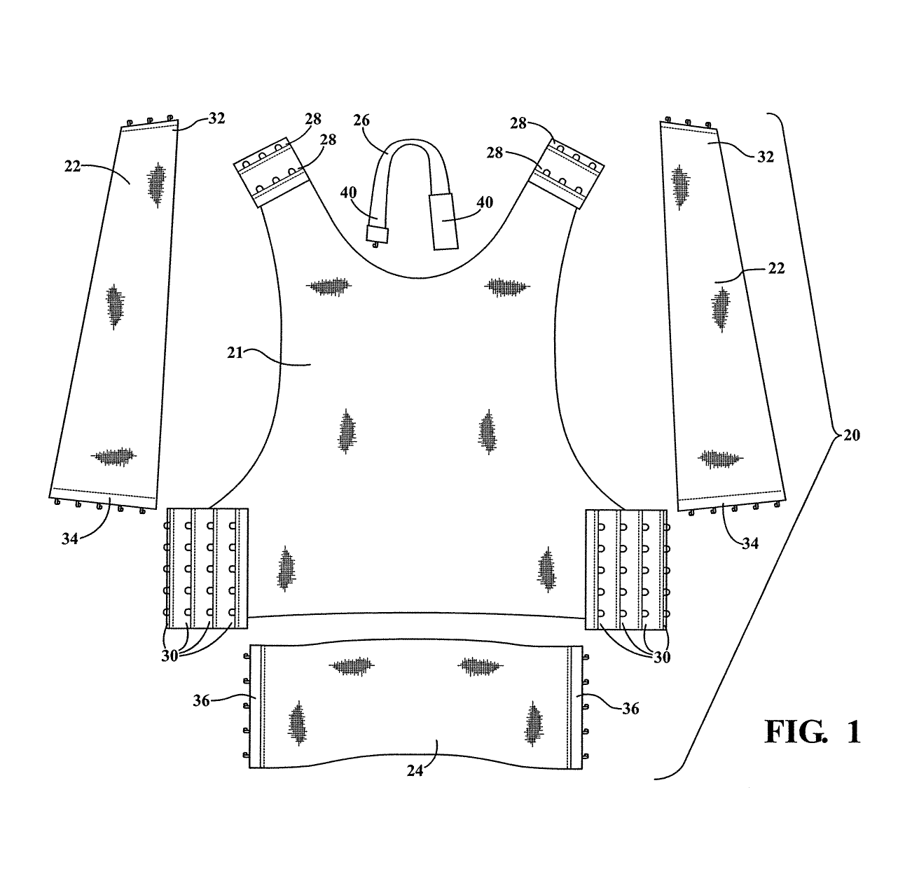

With reference to the attached illustrations, the present invention discloses a breast support garment, the various components of which are depicted generally at 20 in FIG. 1 according to a first exemplary embodiment and include a main body member 21, a pair of sleeves 22, a belt 24 and a fabric strip 26.

The main body member 21 is generally cruciform in shape, having two adjacent ends which each terminate in a number of rows of eye fasteners 28, and another two adjacent ends which terminate in a plurality of rows of eye fasteners 30.

The sleeves 22 can be tubular in configuration, and each have hook fasteners 32 and 34 disposed at opposite ends thereof The belt 24 has a pair of hook fasteners 36 at its ends. The hook fasteners can further be provided as spaced apart sub-pluralities of fasteners as shown or can be reconfigured in any other desired fashion. The fabric strip 26 also has hook and loop fasteners 40 positioned at its ends.

All of the above-described elements can, in one non-limiting embodiment, be constructed out of a fabric sold under the trade-mark LENZING MODAL® by Lenzing AG.

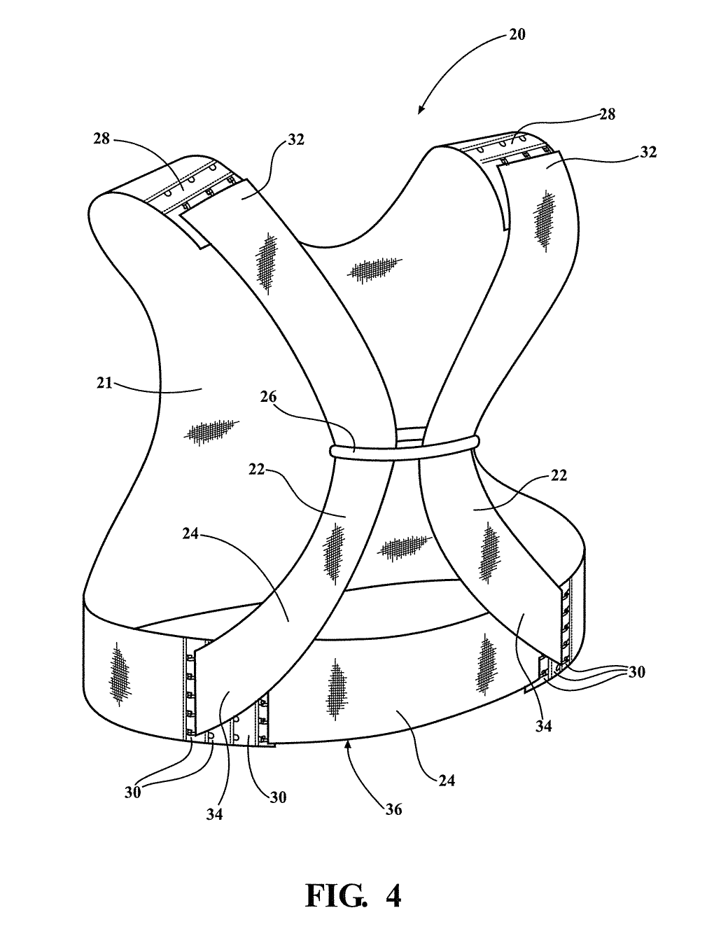

A woman who wishes to wear the garment as a sleeping garment can secure the components as shown in FIG. 4. In this arrangement:
the main body member 21 forms a back side of the garment
the ends of main body member 21 which carry eye fasteners 28 are secured to the ends of the sleeves 22 which carry hook fasteners 32
the hook fasteners 36 of the belt 24 are secured to inner eye fasteners 30 of the main body portion 21
the ends of sleeves 22 which carry hook fasteners 34 are secured to outer eye fasteners 30 of the main body portion 21
hook and loop fasteners 40 are secured to one another to form a loop out of strip 26, which is disposed around sleeves 22

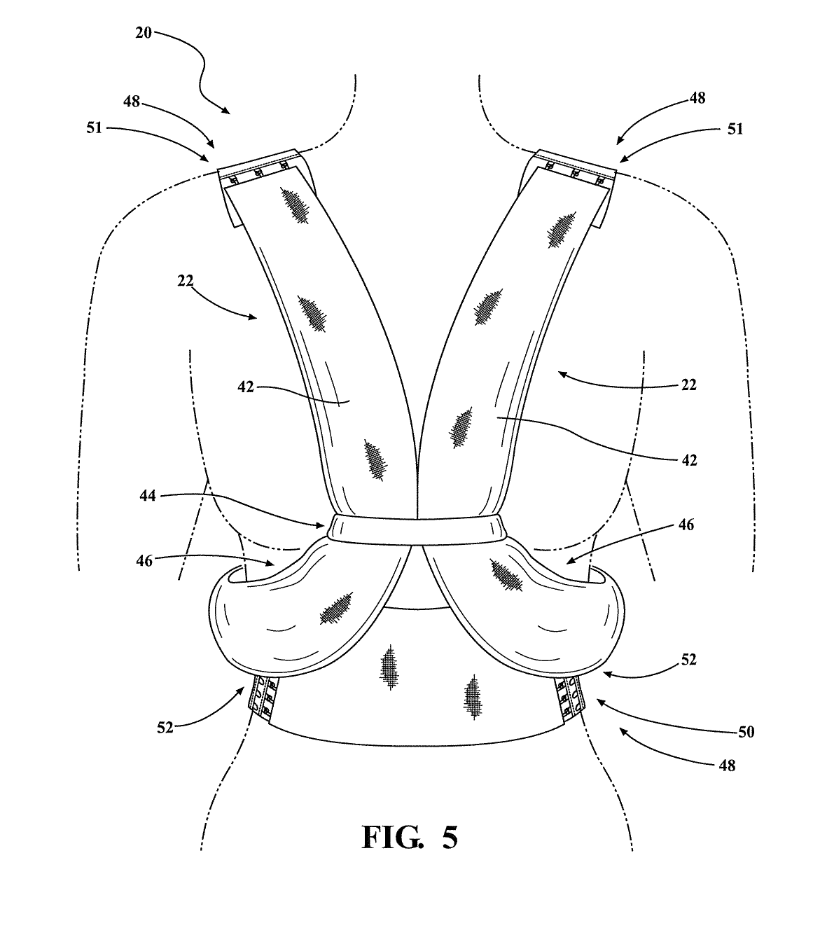

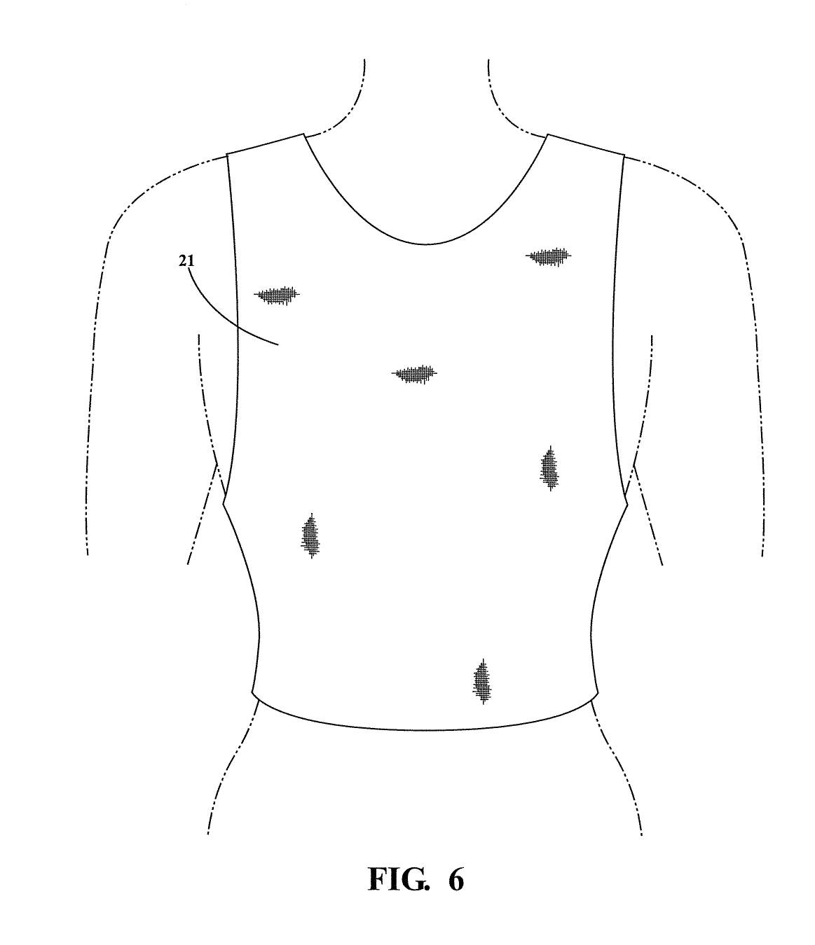

The 20 garment is shown on a mannequin in FIGS. 5-6, and will be seen to include a pair of support portions 46 [defined by portions of the sleeves 22], a central portion 44 [defined by extensions 42 of the support portions 46] and a harness 48. The central portion 44 is disposed between the breasts. The harness 48 includes a girdle portion 50 and a pair of straps 51.

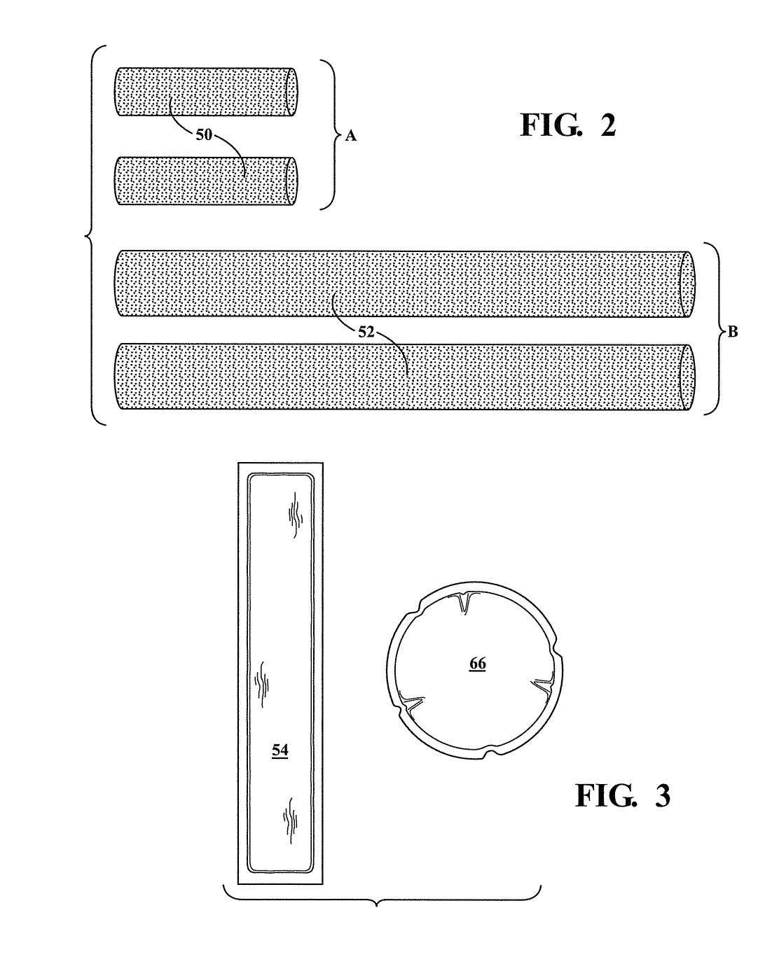

The girdle portion 50 encircles the midriff of the mannequin in a manner which resists upward movement. The straps 51 suspend the central portion 44 and extend from the central portion 44, over the shoulders of the mannequin, to the girdle portion 50. Each of the pair of support portions 46 is disposed beneath a respective one of the breasts and extends from the central portion 44 to the harness 48. As shown, the sleeves 22 have fitted therein elongate cylindrical foam cushions 52 [the cushions being shown in isolation in FIG. 2 and not visible in FIG. 5] which occupy the portions of the sleeves 22 which define the support portions 46 and the extensions 42.

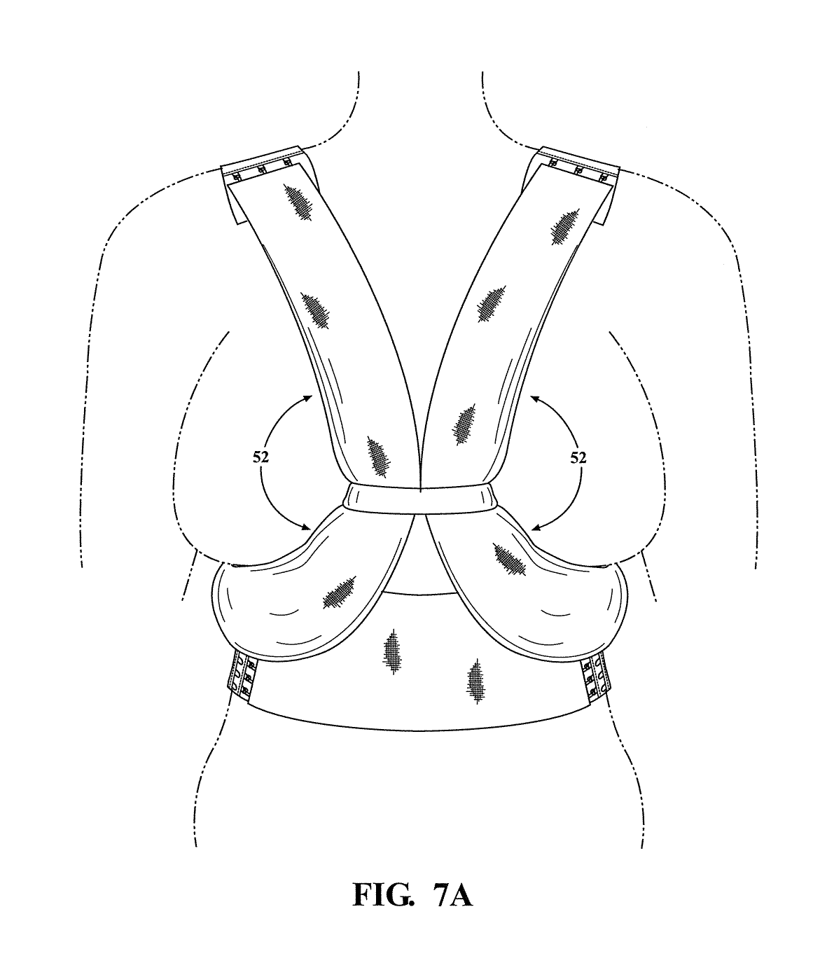

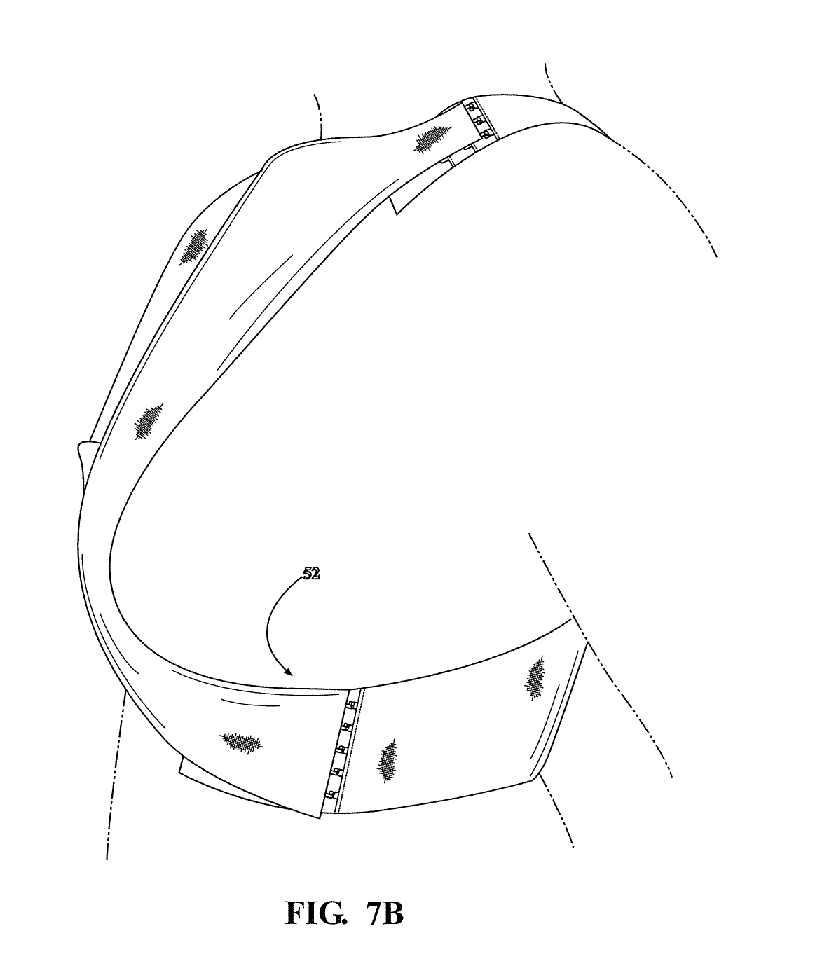

When this garment is disposed on a woman and properly fitted, as shown in FIGS. 7A-7B, cushions 52 support the underside of the breasts, and also extend between the breasts, for lateral support. This configuration of the garment is well-suited for use as a sleeping garment, so as to avoid morning pain that might otherwise follow if a support garment were not worn.

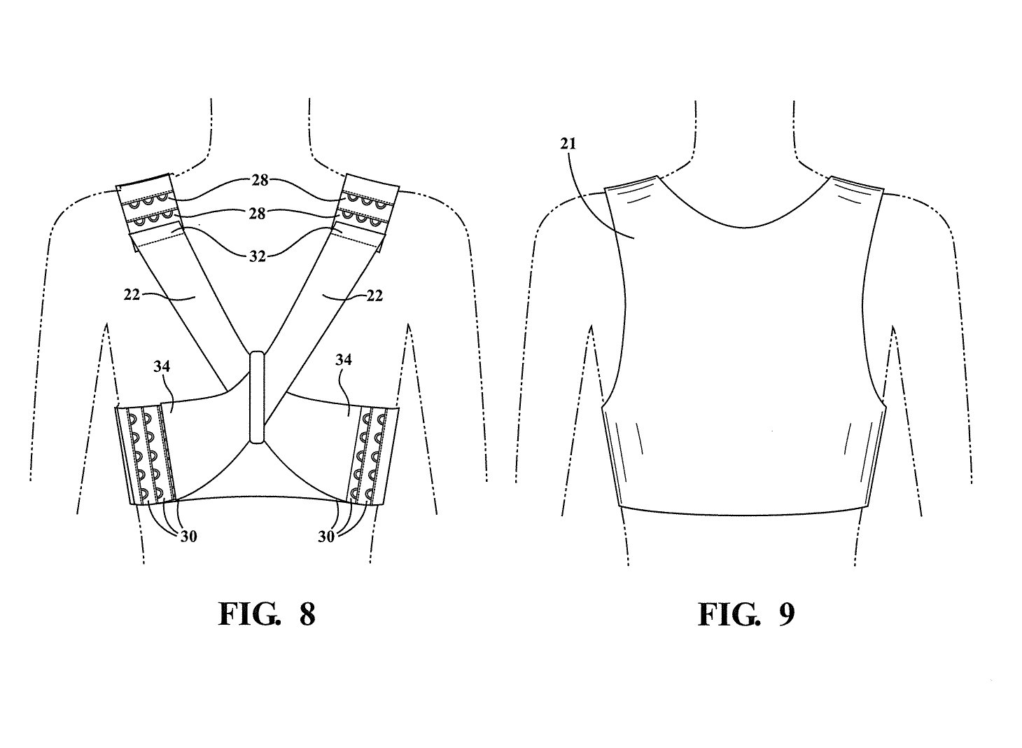

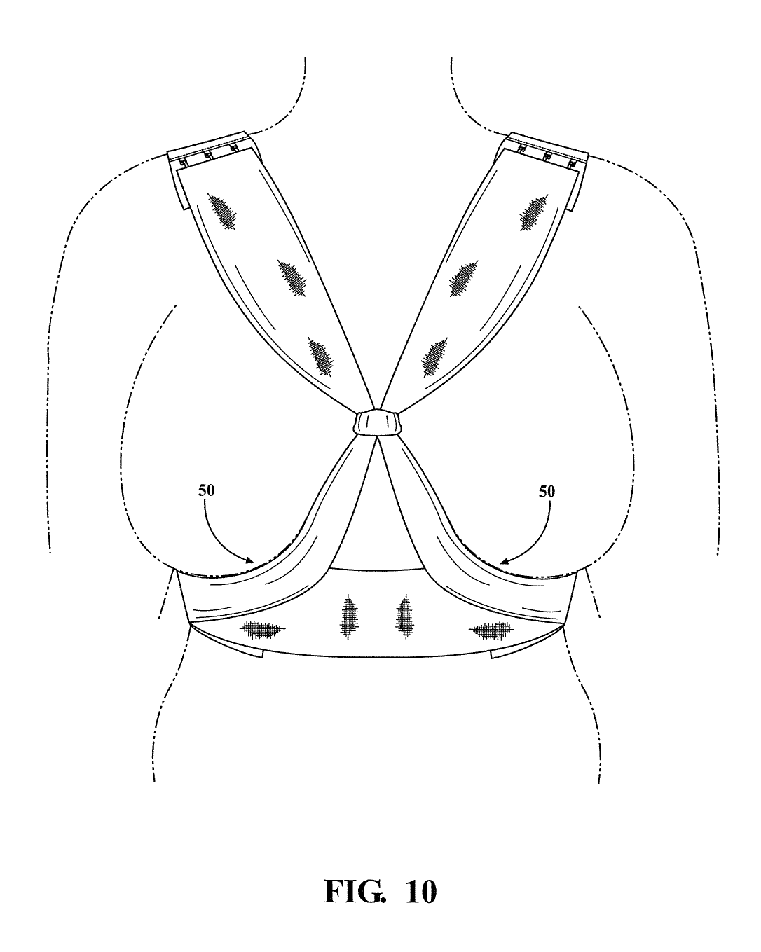

As an alternative, more suitably for daily use, relatively shorter cushions 50, as shown in FIG. 2, may be employed and the sleeves 22 can be arranged in a crossing configuration, as shown in FIGS. 8-9. When this garment configuration is disposed on a woman and properly fitted, as shown in FIG. 10, cushions 50 support the underside of the breasts. This garment can be used in the manner of a conventional brassiere, and provides support without binding. Although this is not necessary for utility, it will be understood that the garment could be worn to great advantage by nursing mothers.

As a result of the plurality of fasteners which characterize the manner in which the various components of the garment are put together, great flexibility is provided, such that a single garment can be used by women of greatly divergent sizes and shapes, as well as different tastes in terms of the amount of breast support provided and girdle tension permitted. As the loop defined by strip 26 is slidable along the spacers 46, flexibility is also provided to accommodate breasts and chests of different shapes.

The cushions 50,52 of the spacer and support portions shown are removable.

Garments according to the present invention can also be used to advantage as part of garment systems.

According to one such system, a gel pack can be provided, to take the place of [or be placed over top of] any one of the cushions. This provides the ability for the garment to be used for providing temperature therapy to the wearer; the gel pack can be heated or cooled as desired, to heat or cool the breast. Such a gel pack is shown in FIG. 3 and indicated with reference numeral 54. The construction of gel packs is well-known, and as such, a detailed description thereof is neither necessary nor provided.

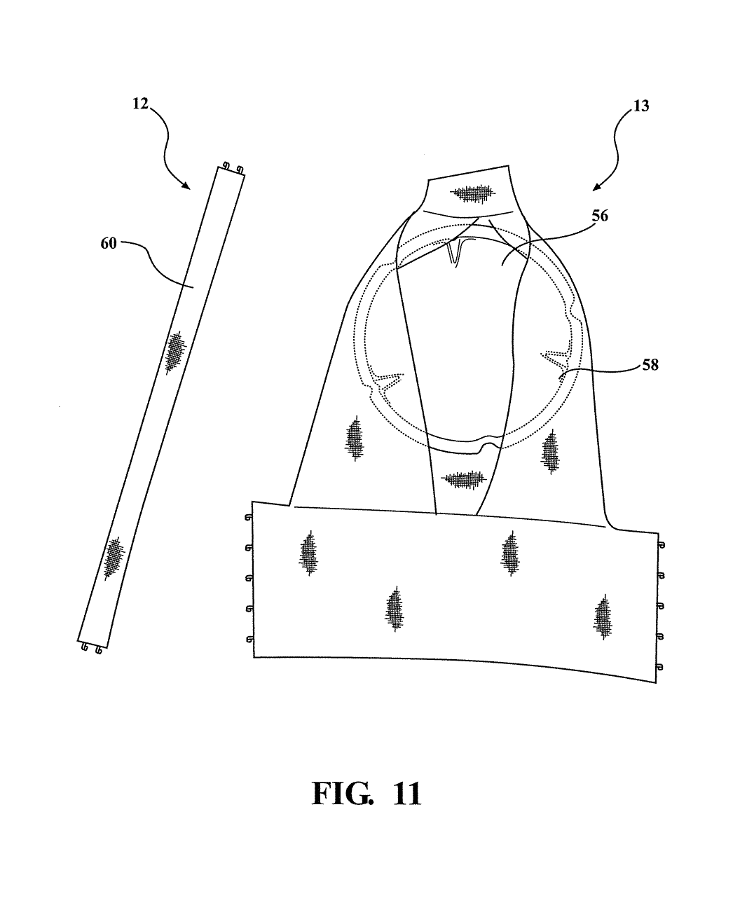

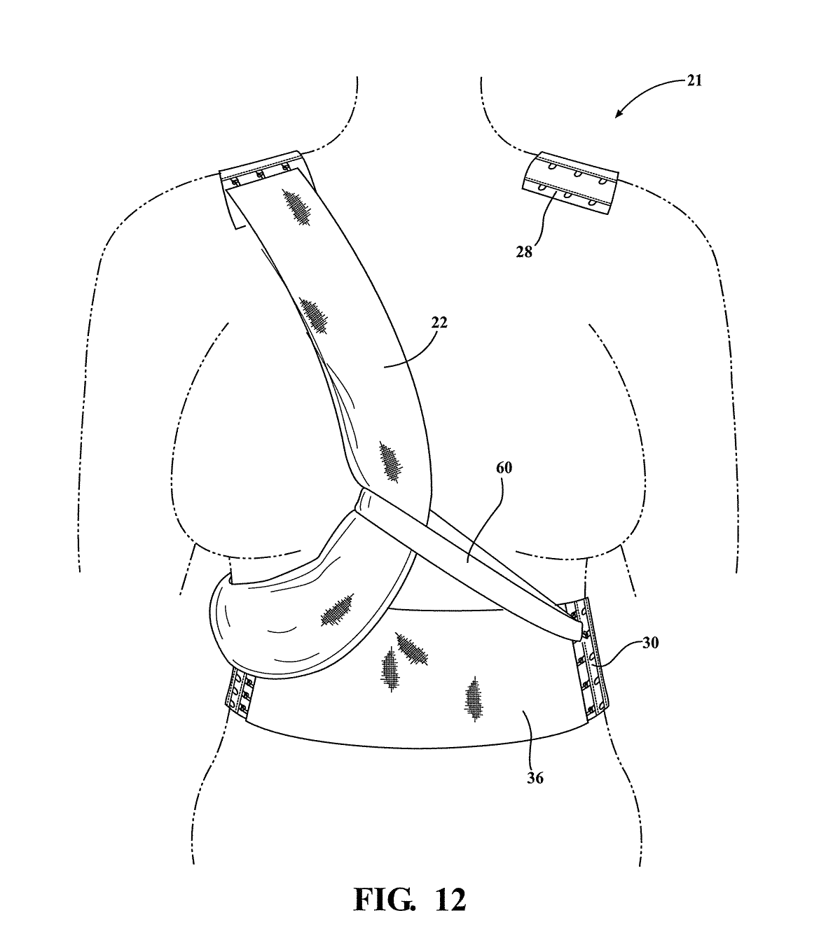

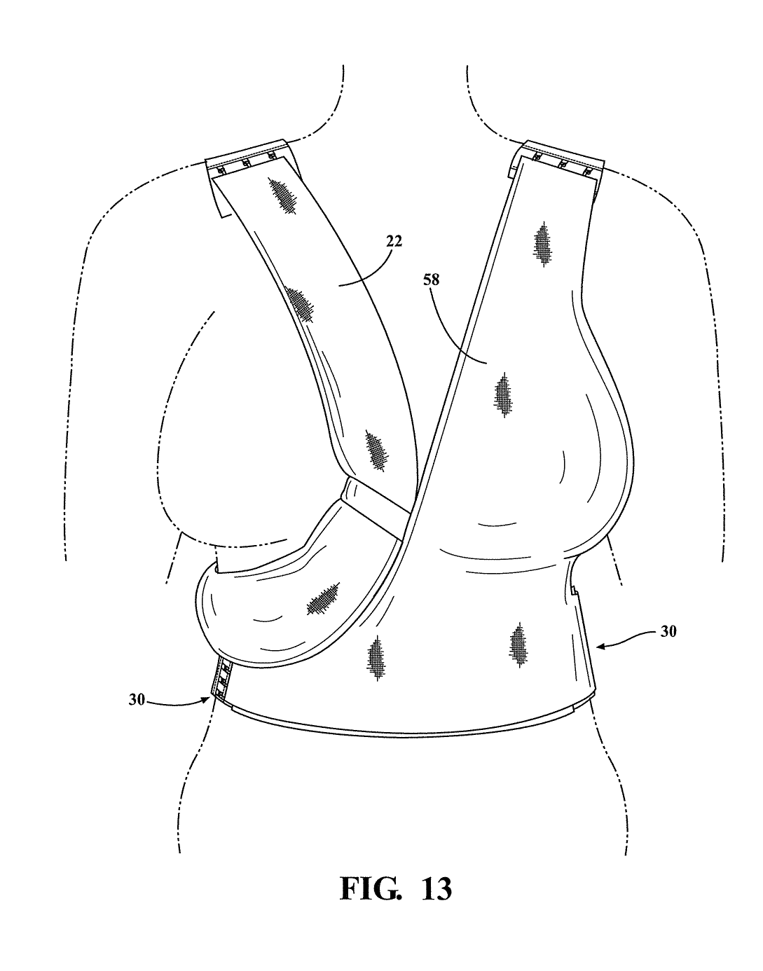

According to another aspect, a prosthetic breast 56, shown in FIG. 11, can be provided, to define one of the breasts of the woman wearing the garment. Also shown in FIG. 11 is a prosthetic holder 58 and a secondary strap 60. As shown in FIG. 12, in circumstances wherein a woman has had a breast removed, the garment may be worn with one of the sleeves 22 removed. The secondary strap 60 is coupled to one of the fasteners 30 of main body portion 21, and the prosthetic holder 58 is secured in place to the eye fasteners 28 of the unused end of the main body portion 21, and to outer eye fasteners 30, all as shown in FIG. 13.

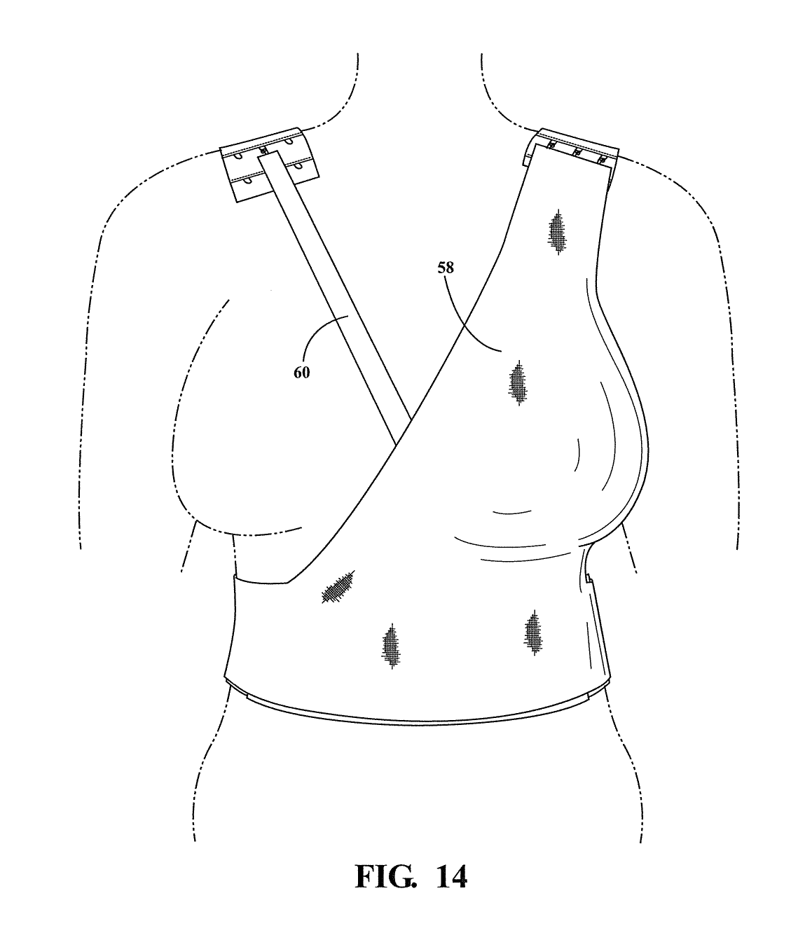

As yet a further variation, the garment can be used as a prosthetic holder, only. In this case, the secondary strap takes the place of one of the sleeves, and the prosthetic holder 58 takes the place of the other of the sleeves, all as shown in FIG. 14.

Other modifications are contemplated. For example, only, foam cushions of different sizes and resilience could be used. As well, the foam cushions could be shaped to enhance comfort and style; for example, the foam cushions could have tapered ends, to better blend-in. Further, whereas in the illustrations, the foams/inserts are removable from the sleeves, which is advantageous from the standpoint of economy in materials, this is not necessary: different sleeves could be provided, each having stitched or otherwise secured therein a respective foam or insert. This would be advantageous in terms of convenience.

As well, other types of fasteners could be employed. Additionally, fabrics other than LENZING MODALS fabric can be employed. Accordingly, the invention should be understood as limited only by the accompanying claims, purposively construed.

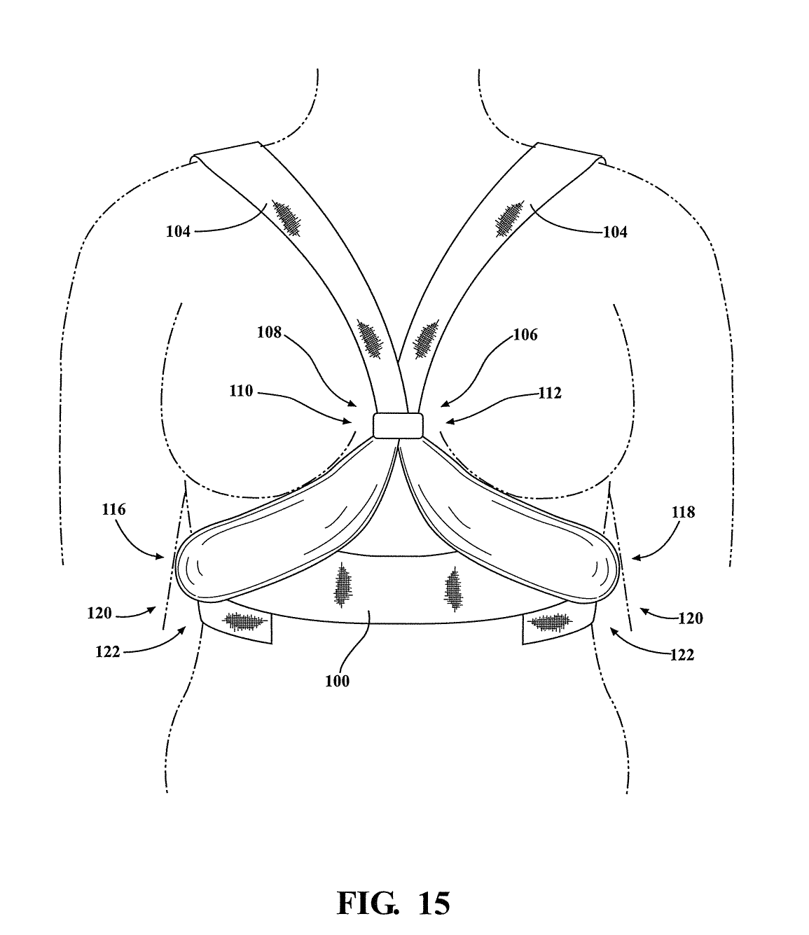

A yet further embodiment of the invention is shown in FIGS. 15-18.

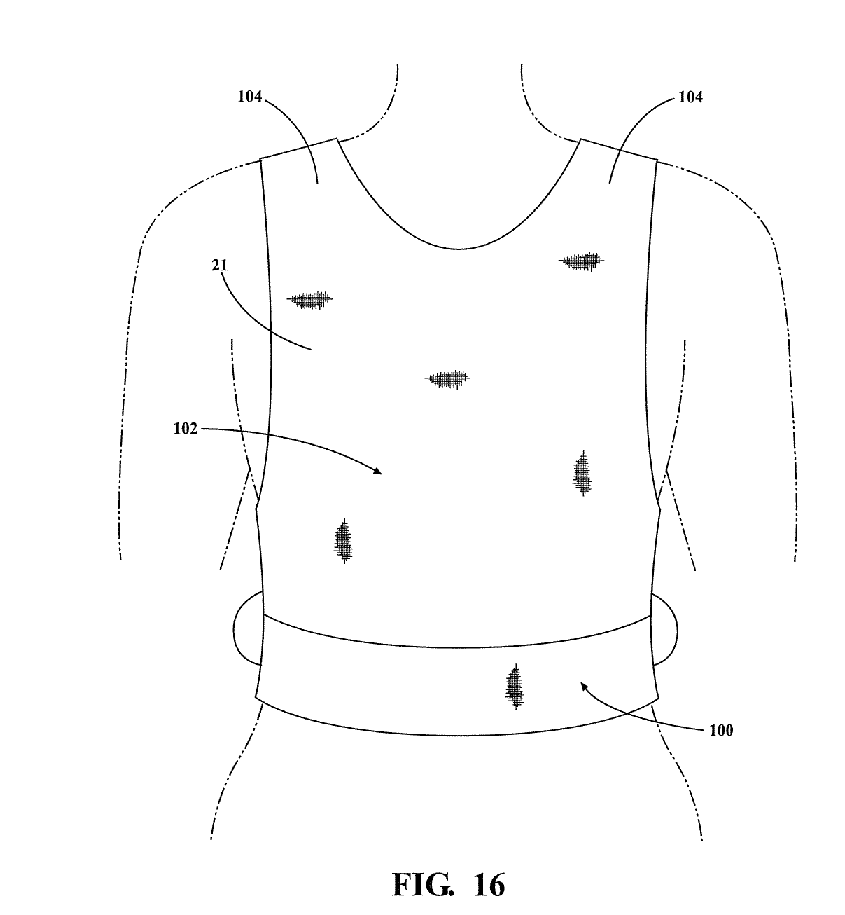

As seen in FIGS. 15 and 16, this embodiment includes a girdle portion 100 encircling the midriff of the wearer to resist upward movement, a main body member 102 forming a back side of the garment, and straps 104 extending over the shoulders, all as generally seen in, for example, FIG. 4.

However, in this embodiment, the straps 104 terminate in a V-neck 106 which carries hook fasteners 108 at the terminus thereof and loop fasteners 110 in spaced relation thereto, to encircle and grasp the central portion 112 of a tubular cushion support 114, the ends 116,118 of the tubular cushion support 114 being secured by hook 120 and loop 122 fasteners to the girdle portion 100 similar to, for example FIG. 5.

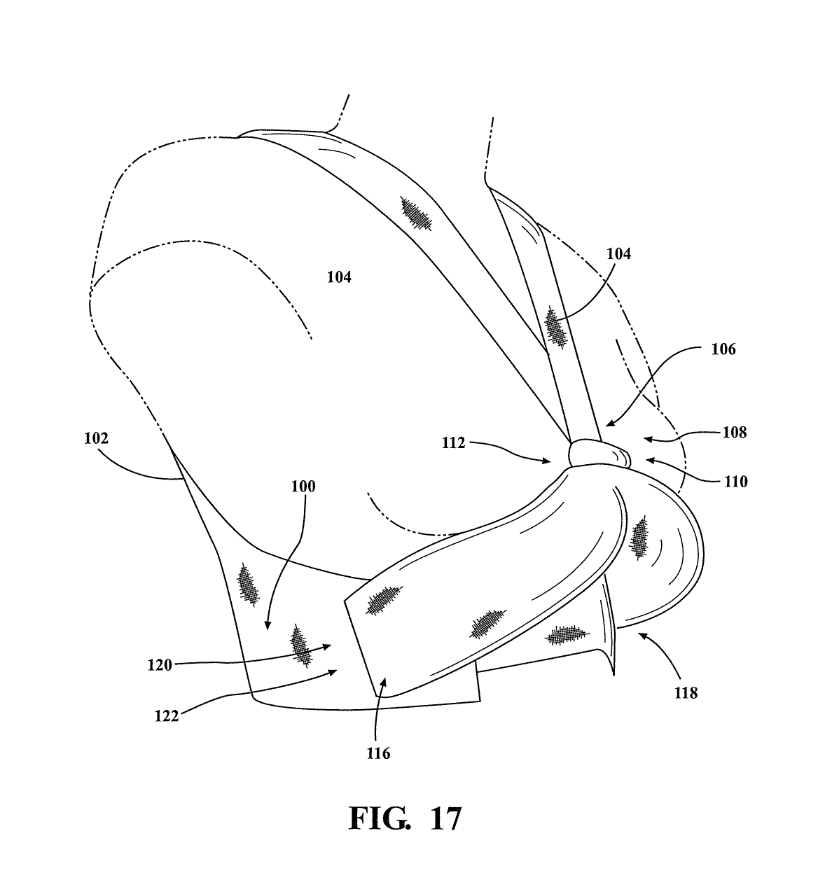

As well, whereas in, for example, FIG. 4, the cushion support was defined by foam, in this embodiment the cushioning for cushion support 114 is provided by an organic granular compressible material, specifically, lavender. The free-flowing nature of the cushioning material allows the cushion 114 to be shaped to fit under the breast, as shown in FIG. 17. This renders the garment less visible in use, and thus renders it more useful for daily use.

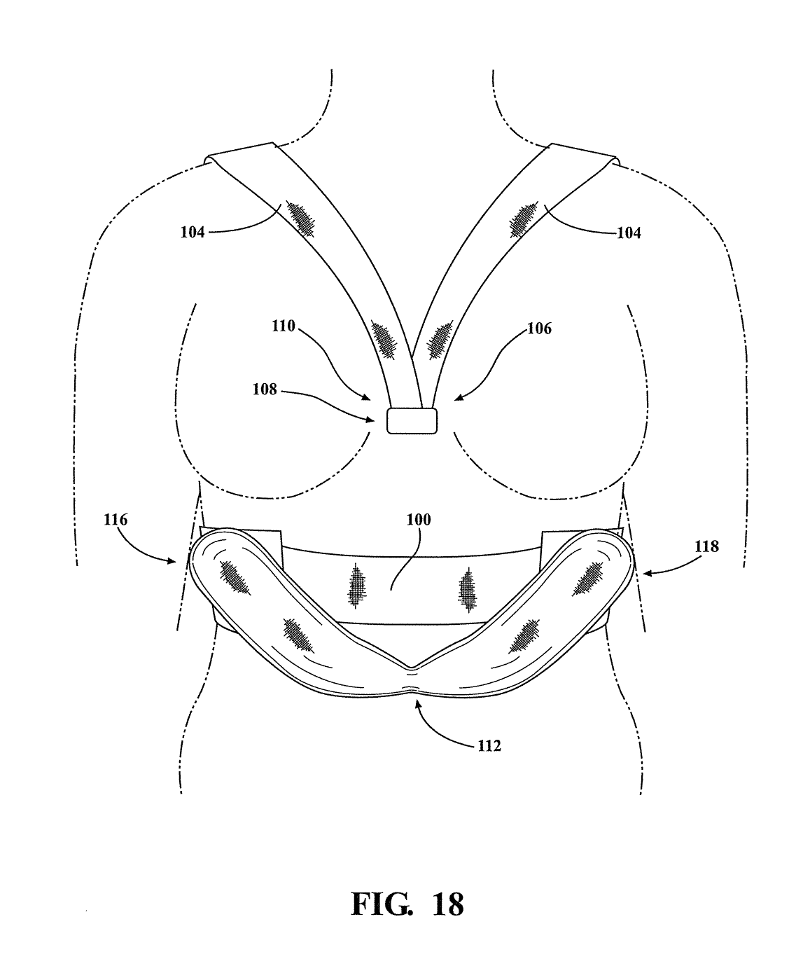

For greater understanding as to the manner in which the central portion 112 of the tubular cushion support 114 is grasped by the V-neck 106, reference can be made to FIG. 18, which shows the garment in a partially disassembled state: here, the ends 116,118 of the tubular cushion support 114 have been secured to the girdle portion 100, but the cushion 114 remains unsecured to the V-neck 106.

Whereas the lavender is specified above for the cushion material, this is not necessary; other organic materials, such as, for example, only, oats, dried beans and other dried flowers can be employed, as can mixtures thereof.

Having described my invention, other and additional preferred embodiments will become apparent to those skilled in the art to which it pertains, and without deviating from the scope of the appended claims.

I claim:

1. A garment for use by a woman having a pair of breasts, the garment comprising:
a central portion which is adapted to being disposed between the pair of breasts in use;
a harness, including:
a girdle portion which encircles a midriff of the woman in use in a manner which resists upward movement; and
a pair of straps from which the central portion is suspended in use and which, in use, is adapted to extend from the central portion, over the shoulders of the woman, to the girdle portion; and
a pair of support portions, each adapted to being disposed beneath a respective one of said pair of breasts in use and extending from the central portion to the harness.

2. The garment according to claim 1, the central portion further comprising is a pair of spacers, each spacer being defined by an extension of a respective support portion.

3. The garment according to claim 2, further comprising the spacers being secured together in use.

4. The garment according to claim 2, further comprising the support portions being cylindrical and each spacer is defined by a cylindrical extension of a respective support portion.

5. The garment according to claim 1, further comprising the central portion being cushioned and adapted to space the breasts apart from one another.

6. The garment according to claim 2, further comprising the support portions being cushioned and adapted to support the undersides of the breasts.

7. The garment according to claim 1, further comprising the central portion being cushioned and adapted to space the breasts apart from one another, the support portions also being cushioned and adapted to support the undersides of the breasts.

8. The garment according to claim 7, further comprising the cushions being removable from the central and support portions for laundering of the garment.

9. The garment according to claim 7, further comprising at least one of the cushions associated with the central and support portions being cylindrically shaped.

10. A system incorporating a garment for use by a woman having a pair of breasts, comprising:
the garment having a central portion which is adapted to being disposed between the pair of breasts in use;
a harness, including:
a girdle portion which is adapted to encircle a midriff of the woman in use in a manner which resists upward movement; and
a pair of straps from which the central portion is suspended in use and which, in use, extend from the central portion, over the shoulders of said woman, to the girdle portion;
a pair of support portions, each adapted to being disposed beneath a respective one of said pair of breasts in use and extending from the central portion to the harness;
the central portion including a cushion and adapted to space the breasts apart from one another, the support portions also each including a cushion and adapted to support the undersides of the breasts; and
a gel pack adapted to be substituted for at least one of the cushions, for providing temperature therapy to the wearer.

11. A system incorporating a garment for use by a woman having a pair of breasts, comprising:
the garment having a central portion which is adapted to being disposed between the pair of breasts in use;
a harness, including:
a girdle portion which is adapted to encircle a midriff of the woman in use in a manner which resists upward movement; and
a pair of straps from which the central portion is suspended in use and which, in use, is adapted to extend from the central portion, over the shoulders of said woman, to the girdle portion;
a pair of support portions, each adapted to being disposed beneath a respective one of said pair of breasts in use and extending from the central portion to the harness; and
a prosthetic which defines one of the breasts of the woman in use and which is suspended from the harness.
